SEQUENCE LISTING (9 sequences)

SEQ ID NO: 1 - DNA, 20 bp, Artificial Sequence
  Synthetic
atcagatgacatagatatat

SEQ ID NO: 2 - DNA, 19 bp, Artificial Sequence
  Synthetic
atagatcgatgatgaggac

SEQ ID NO: 3 - protein, 82 aa, Artificial Sequence
  Synthetic
LAKLVLNSLYGKFASNPDVTGKVPYLKENGALGFRLGEEETKDPVYTPMGVFITAWARYTTITAAQACYDRIIYCDTDSIHL

SEQ ID NO: 4 - protein, 82 aa, Artificial Sequence
  Synthetic
MAKLVLNSLYGKFGTSIDVTGKEVFLKEDGSTGFRKGQKEERDPVYMPMGAFITAYARDVTIRTAQKCYDRILYCDTDSIHL

SEQ ID NO: 5 - protein, 82 aa, Artificial Sequence
  Synthetic
LAKLQLNSLYGKFASHPDVTGKVPYLKDDGSTAFKKGLPKSKDPVYTPAGAFITAWARHMTITTAQKVYDRILYCDTDSIHI

SEQ ID NO: 6 - protein, 82 aa, Artificial Sequence
  Synthetic
LAKLMFDSLYGKFASNPDVTGKVPYLKEDGSLGFRVGDEEYKDPVYTPMGVFITAWARFTTITAAQACYDRIIYCDTDSIHL

SEQ ID NO: 7 - protein, 82 aa, Artificial Sequence
  Synthetic
NAKGMLNSLYGKFGTNPDITGKVPYMGEDGIVRLTLGEEELRDPVYVPLASFVTAWGRYTTITTAQRCFDRIIYCDTDSIHL

SEQ ID NO: 8 - protein, 82 aa, Artificial Sequence
  Synthetic
QAKLMLNSLYGKFATNPDITGKVPYLDENGVLKFRKGELKERDPVYTPMGCFITAYARENILSNAQKLYPRFIYADTDSIHV

SEQ ID NO: 9 - protein, 81 aa, Artificial Sequence
  Synthetic
IAKLHLNSLYGKFASNPNVTSKIPILKDGIVKLVRGVPEQRPPVYTAAGVFITAYARNITIRAAQANFDSFAYADTDSLHL

(19) United States
(12) Patent Application Publication
(10) Pub. No.: US 2021/0202041 A1
(43) Pub. Date: Jul. 1, 2021

(54) PROTEIN HOMOLOG DISCOVERY

(71) Applicant: Homodeus, Inc., Guilford, CT (US)

(72) Inventors: Harry Kemble, Paris (FR); Spencer Glantz, West Hartford, CT (US); Jonathan M. Rothberg, Guilford, CT (US)

(73) Assignee: Homodeus, Inc., Guilford, CT (US)

(21) Appl. No.: 17/118,172

(22) Filed: Dec. 10, 2020

Related U.S. Application Data
(60) Provisional application No. 62/946,179, filed on Dec. 10, 2019.

(51) Int. Cl. G16B 35/10; G16B 30/10; G06N 7/00

(52) U.S. Cl. CPC G16B 35/10; G06N 7/00; G16B 30/10

(57) ABSTRACT

The present disclosure provides, in some aspects, protein homolog discovery methods for enhanced co-evolution-based protein structure prediction.

### RELATED APPLICATION

This application claims the benefit under 35 U.S.C. § 119(e) of U.S. provisional application No. 62/946,179, filed Dec. 10, 2019, which is incorporated by reference herein in its entirety.

### BACKGROUND

Proteins are macromolecules that are comprised of strings of amino acids, which interact with each other and fold into complex three-dimensional shapes with characteristic structures. Many in silico analyses of protein structure and function begin by identifying a protein's “homologs.” Two proteins are considered homologous if they are descended from a common ancestor.

### SUMMARY

Provided herein, in some aspects, are methods for training and executing co-evolution based structural prediction models based on a protein homolog discovery platform technology.

Some aspects of the present disclosure provide methods of in silico mining for new homologs of a protein of interest, the method comprising producing an initial protein homolog sequence database (DBinit) for the protein of interest; generating a representative reference database (DBrep) of putative protein homolog sequences by eliminating multiple sequences in the DBinit that share at least 75% identity; screening a metagenomic read database using the DBrep as a query to identity datasets of sequencing reads, and optionally ranking the datasets to determine which are most likely to contain the highest number of true homologs; aligning the DBrep to sequencing reads of the metagenomic datasets; assembling the sequencing reads into contigs (a set of overlapping DNA segments that together represent a consensus region of DNA); translating open reading frames (ORFs) of the contigs into protein sequences having greater than a cutoff fraction of the length of the average DBrep protein sequence; aligning the translated protein sequences with the DBrep protein sequences and identifying new putative protein homolog sequences, and optionally adding the new putative protein homolog sequences to the DBinit to produce an enhanced protein homolog sequence database (DBenhanced). In some embodiments, the whole-genome metagenomic fraction of the NCBI sequencing read archive (SRA) is the metagenomic read archive that is screened using DBrep as a query.

Other aspects of the present disclosure provide computer implemented methods of mining for new homologs of a protein of interest, the method comprising: producing an initial protein homolog sequence database (DBinit) for the protein of interest; generating a representative reference database (DBrep) of putative protein homolog sequences by eliminating multiple sequences in the BDinit that share at least 75% identity; screening a whole-genome metagenomic sequencing read database using the DBrep as a query to identify datasets of sequencing reads, and optionally ranking the datasets to determine which are most likely to contain the highest number of true homologs; aligning the DBrep to sequencing reads of the whole-genome metagenomic datasets; optionally assembling sequencing reads that are shorter than a full-length sequence of the protein of interest into contigs; translating open reading frames (ORFs) of long sequencing reads and/or assembled contigs into protein sequences having greater than a cutoff fraction of the length of the average DBrep protein sequence; aligning the translated protein sequences with the DBrep protein sequences and identifying new putative protein homolog sequences, and optionally adding the new putative protein homolog sequences to the DBinit to produce an enhanced protein homolog sequence database (DBenhanced).

In some embodiments, producing a protein homolog sequence database includes searching protein family databases for proteins containing a conserved protein domain. In some embodiments, producing a protein homolog sequence database includes searching protein sequence databases using pairwise or hidden Markov model (HMM)-based alignment.

In some embodiments, the methods further comprise assessing completeness of the DBinit by aligning a known non-redundant protein reference database and the DBinit, optionally using a protein alignment tool adapted for large query sets and searching for additional homologs of the protein of interest.

In some embodiments, the DBrep is generated by clustering the DBinit at 90% using a clustering algorithm.

In some embodiments, aligning the DBrep to sequencing reads of whole-genome metagenomic datasets in a read archive comprises aligning the DBrep to a sampling of reads/read-pairs from each individual whole-genome metagenomic run, optionally wherein the sampling size is about 100,000 reads.

In some embodiments, the methods further comprise quality control steps to remove unassembled reads from the sequencing read datasets.

In some embodiments, translating comprises translating six ORFs of the contigs.

In some embodiments, the methods further comprise quality control steps to validate the putative protein homolog sequences as true protein homolog sequences, which are then optionally added to the DBenhanced.

In some embodiments, the methods further comprise target protein enrichment.

In some embodiments, the methods further comprise generating a representative multiple sequence alignment (MSA) based on the DBenhanced.

Other aspects of the present disclosure provide target enrichment methods comprising: providing a list of putative protein homolog sequences of a protein of interest from a multiple sequence alignment (MSA) of sequences homologous to the protein of interest; contacting a sample comprising DNA with probes to produce probes bound to DNA, wherein the probes are designed to hybridize, optionally with low stringency, to the nucleotide sequences of the putative protein homolog sequences, and wherein the probes are immobilized on a substrate that optionally includes a separation medium; selectively removing from the substrate probes that are not bound to DNA; sequencing the DNA bound to the probes to produce sequencing reads; aligning the sequencing reads to the MSA and assembling contigs from any sequencing reads that are shorter than the full-length sequence of the protein; translating open reading frames (ORFs) from the contigs to generate new putative protein homolog sequences, and optionally validating the new putative protein homolog sequences as true protein homolog sequences; and optionally adding the new putative protein homolog sequences to the MSA to produce an enriched MSA.

In some embodiments, the methods further comprise executing on the MSA an algorithm for deducing direct correlation, optionally wherein the algorithm is a Direct Coupling Analysis (DCA) algorithm.

In some embodiments, the methods further comprise performing feature extraction using the enriched MSA for a co-evolution-based protein structure prediction model.

Further aspects of the present disclosure provide iterative homolog discovery methods comprising: (a) performing a method of in silico mining for new homologs of a protein of interest to produce an enhanced multiple sequence alignment (MSA) as described herein; (b) performing a target enrichment method as described herein to identify new putative protein homolog sequences, wherein the DNA sample has been identified using metadata for metagenomic SRA samples with positive homolog identification; (c) adding the new putative protein homolog sequences to the enhanced MSA; and optionally repeating the steps (a)-(c) iteratively.

Some aspects of the present disclosure provide computer implemented iterative homolog discovery methods comprising: (a) performing a method of in silico mining for new homologs of a protein of interest to produce an enhanced multiple sequence alignment (MSA) as described herein; (b) processing new putative protein homolog sequences obtained by a target enrichment method as described herein, wherein the DNA sample has been identified using metadata for metagenomic SRA samples with positive homolog identification; (c) adding the new putative protein homolog sequences to the enhanced MSA; and optionally repeating the steps (a)-(c) iteratively.

Also provided herein is a computer readable medium on which is stored a computer program which, when implemented by a computer processor, causes the processor to: produce an initial protein homolog sequence database (DBinit) for the protein of interest; generate a representative reference database (DBrep) of putative protein homolog sequences by eliminating multiple sequences in the DBinit that share at least 75% identity (e.g., at least 80% or at least 90% identity); screen a whole-genome metagenomic sequencing read archive using the DBrep as a query to identity datasets of sequencing reads, and optionally rank the datasets to determine which are most likely to contain the highest number of true homologs.

In some embodiments, the computer program further causes the processor to: align the DBrep to sequencing reads of the metagenomic datasets to identify hit reads; assemble hit reads into contigs; translate open reading frames (ORFs) of the contigs into protein sequences having greater than a cutoff fraction of the length of the average DBrep protein sequence; align the translated protein sequences with the DBrep protein sequences and identifying new putative protein homolog sequences, and optionally add the new putative protein homolog sequences to the DBinit to produce an enhanced protein homolog sequence database (DBenhanced).

Additional aspects of the present disclosure provide a computer readable medium on which is stored a computer program which, when implemented by a computer processor, causes the processor to: align sequencing reads to a multiple sequence alignment (MSA) and assembling contigs from any sequencing reads that are shorter than a full-length sequence of the protein; translating open reading frames (ORFs) from the contigs to generate new putative protein homolog sequences; and add the new putative protein homolog sequences to the MSA to produce an enriched MSA.

### BRIEF DESCRIPTION OF THE DRAWINGS

FIG. 1 is a flow diagram of the steps of an illustrative process for discovering protein homologs.

FIGS. 2A-2B are flow diagrams showing steps 1 (FIG. 2A) and 2 (FIG. 2B) of an example methodology for in silico Phi29 homolog mining from the whole-genomic metagenomic fraction of the NCBI Sequence Read Archive (SRA).

FIG. 3 is a flow diagram of the steps of an illustrative process for probe design.

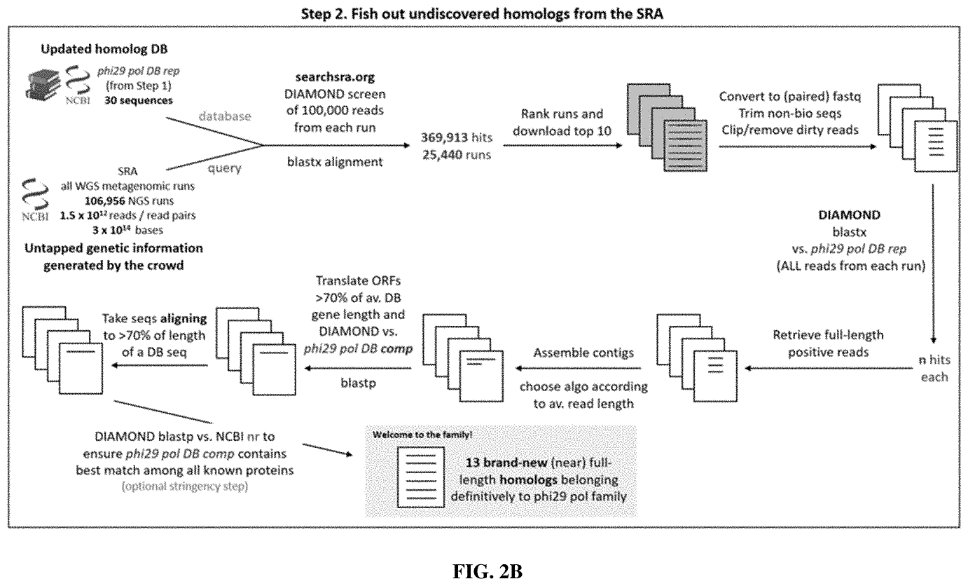

FIG. 4 is a schematic showing construction of a representative reference MSA for the 16S gene.

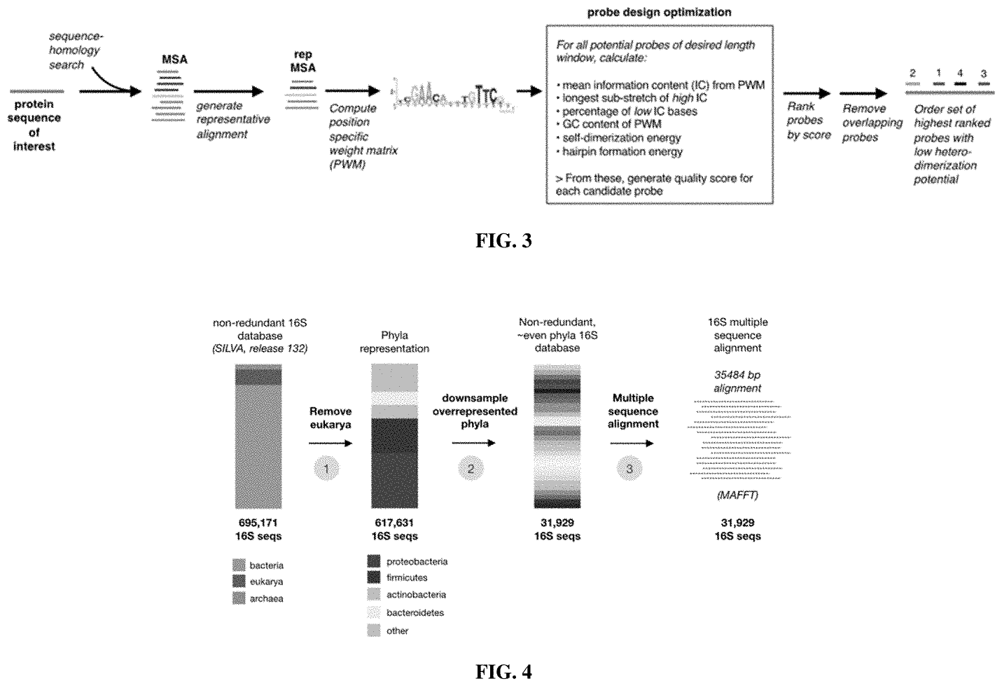

FIG. 5 includes graphs representative of an associated position-specific weight matrix (PWM) for the 16S gene example.

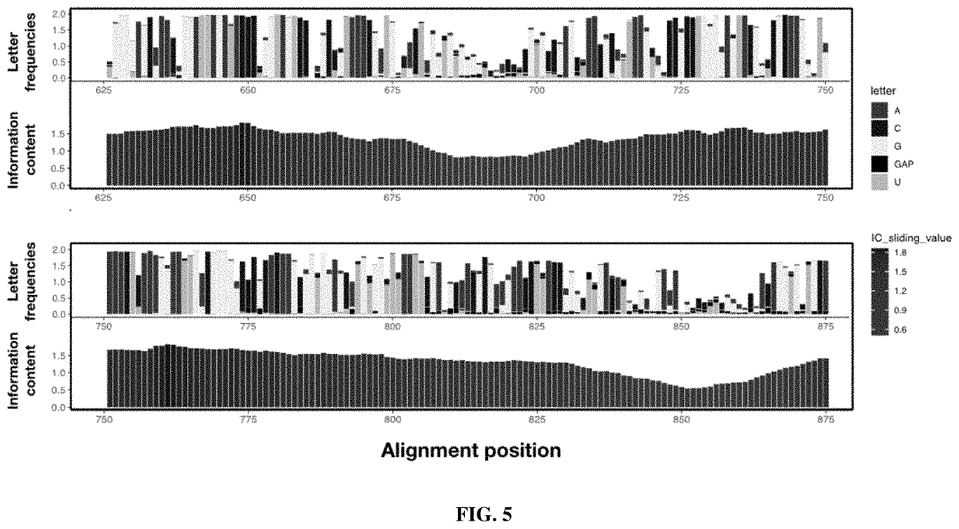

FIG. 6 is a flow diagram of the steps for candidate probe scoring and ranking for the 16S gene example.

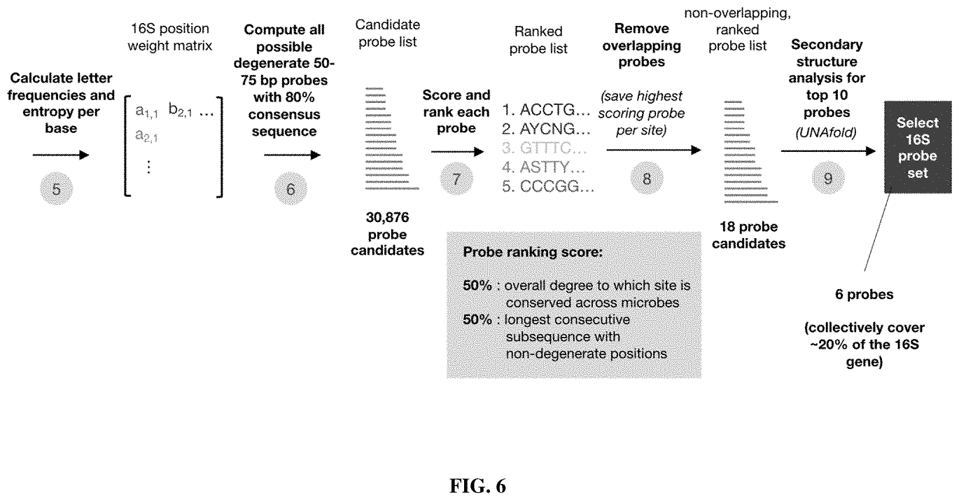

FIG. 7 is an alignment showing a selected optimal probe set for the 16S gene. Designed optimal probes overlap with conserved regions identified by others as optimal probe regions.

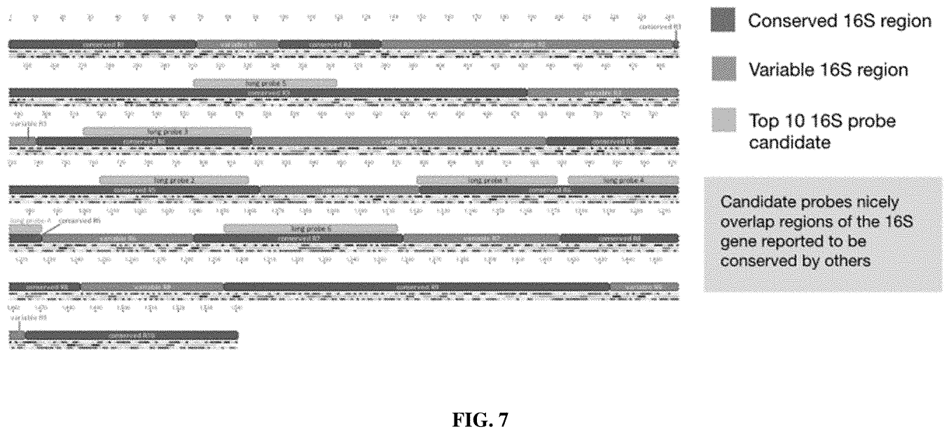

FIG. 8 is an example fragment length distribution for a tagmented soil library.

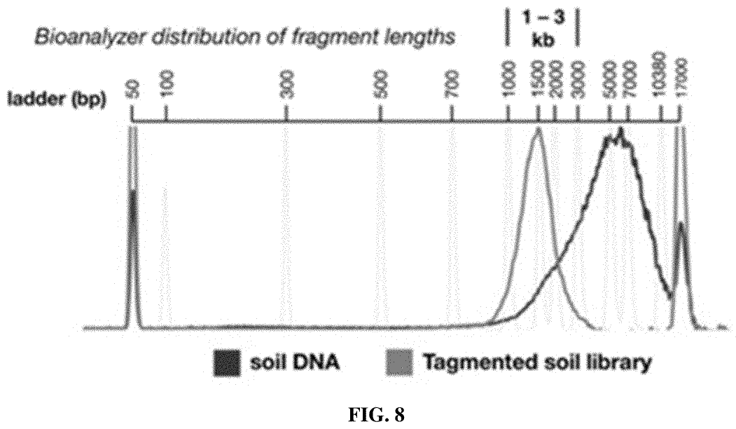

FIG. 9 includes graphs showing the results of tuning scodaphoresis parameters to control the stringency of target enrichment.

FIG. 10 is a flow diagram of the overall workflow for the example application, target enrichment by scodaphoresis.

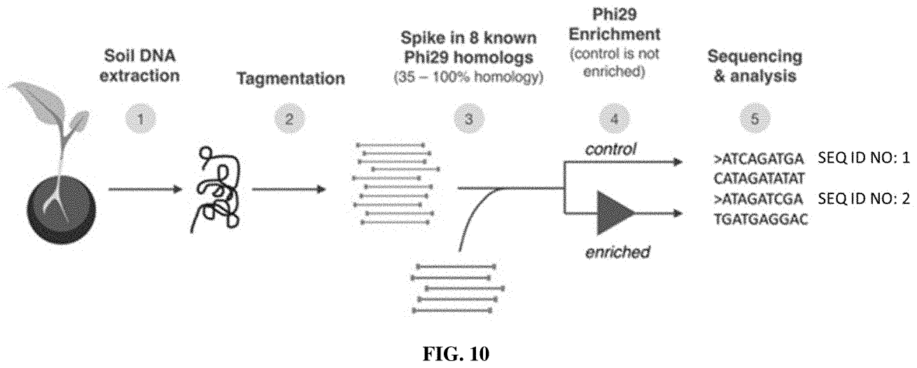

FIG. 11 is a diagram of the scodaphoresis methodologies implemented.

FIG. 12 includes graphs showing read length statistics for pre- and post-enriched soil samples.

FIG. 13 includes graphs showing protein domain frequency in the pre and post-enriched samples.

FIG. 14A includes graphs showing quantification of enrichment across scodaphoresis methods at individual homolog level.

FIG. 14B includes graphs showing a comparison of DM and OT scodaphoresis approaches for mining divergent sequences.

FIG. 15 is a description and sample alignment of the new OT_102800 homolog.

FIG. 16 is an updated phylogeny of the Phi29 family with the newly discovered OT_102800 homolog.

FIG. 17 is a block diagram of an illustrative implementation of a computer system for discovering protein homologs.

### DETAILED DESCRIPTION

Protein engineering is the process of modifying a protein by altering its chemistry, usually to improve its function for a particular application. Proteins are biological machines with many industrial and medical applications; proteins are used in detergents, cosmetics, bioremediation, the catalysis of industrial-scale reactions, life science research, agriculture, and the pharmaceutical industry, with many modern drugs derived from engineered recombinant proteins.

Solving structures of existing proteins can be a fundamental step in engineering new proteins as it provides a three-dimensional (3D) map of the protein's chemistry. The structure can be used to identify target amino acid residues that are most likely to influence protein function. Mutation of these amino acids leads to the creation of new protein variants, some of which will have enhanced properties. Identifying these key amino acids is a useful step for rational design of proteins and for some variations of directed evolution, including site-directed mutagenesis. Beyond its application for engineering new proteins, successful protein structure prediction can be used to better understand the structure/function of known, existing proteins, relevant to basic science, drug discovery, biotechnology, and a number of field applications.

Traditionally, protein structures have been generated from empirical data sourced from quantitative experimental measurements. More recently, structural prediction has been made possible by in silico modeling. Due to the inherent challenges and limitations of existing methods for empirically elucidating protein structure, such as X-ray crystallography and NMR spectroscopy, Applicants had an interest in developing software that could determine a protein's structure from its amino acid sequence. In silico analyses of protein structure and function can begin by identifying a protein's “homologs.” Two proteins are considered homologous if they are descended from a common ancestor. Homologous proteins can have substantially different sequences, but they often have similar function and structure. Once a protein of interest's homologs are known, there are several possible in silico routes to protein structure prediction.

In some cases, a 3D structure is not available for the protein of interest, but a 3D structure has already been experimentally gathered for an identified homolog. Because similar amino acid sequences adopt similar structures, an amino acid sequence alignment of the target protein and the homolog as well as the experimentally determined homolog's structure can be used to generate an atomic model of the target protein. This process is called “homology modeling.” If a full-length homologous protein with known structure cannot be found, one can also look for homology between small subsets of the target protein and libraries of shorter homologous sequences, each of which adopt a known fold. This “protein threading” approach can thus be used to build a structure from a collection of short homologous sequences, each contributing to defining a portion of the overall structure.

If a protein of interest has no suitable homologous templates, ab initio methods may be used to predict the structure of the protein from amino acid sequences alone. Ab initio methods include physics-based modeling, where thermodynamic and molecular energy parameters are used to propose and rank candidate structures until a minimum entropy/maximum stability model is found.

It is also possible to infer information about a protein's three-dimensional structure by comparing the sequences of homologs and measuring the correlations in amino acid identity at pairs of residues. If two non-neighboring residues are physically in contact, for example by forming a hydrogen bond, then the amino acid identities in these positions will be correlated. Should a mutation at one position occur, it will likely be accompanied by a compensatory mutation in the other residue. In contrast, for two non-neighboring residues that are not in contact, there is less likely to be a correlation between their amino acid identities. Co-evolutionary statistical models that capture the tendency of particular pairs of residues to mutate together within a family of protein homologs can thus be used to generate “contact maps” that describe inter-residue contacts protein-wide. Contact maps are an important first step towards predicting all inter-residue (pairwise) distances for the amino acids in a protein. Such a distance matrix would be completely descriptive of the 3D structure, and thus, contact maps are an important element of computational protein structure prediction.

Direct Coupling Analysis

When generating contact map predictions, Applicants have recognized that the analysis should go beyond the raw correlations, due to the fact that some observed correlations may be indirect. For example, if residue A interacts with residue B, and residue B interacts with residue C, there will be a substantial correlation between residues A and C, but no true contact between A and C. To leverage co-evolutionary data for accurate structural determination, it is helpful to distinguish direct and indirect correlations. One algorithm for deducing direct correlations is Direct Coupling Analysis (DCA). Once a collection of all the known protein sequences that are homologous to a protein of interest have been assembled into a multiple sequence alignment (MSA), direct coupling analysis (DCA) can be performed to solve a Potts model on the alignment. The output of DCA is a matrix that represents the “strength” of the coupling between all pairs of residues. Empirically, it has been demonstrated that a high DCA output value often indicates that the two residues are physically in contact. The quality of the DCA analysis is measured by the extent to which the output, when thresholded appropriately, produces accurate predictions for whether or not each pair of residues is in contact (defined by being within a certain distance from each other).

Applicants have appreciated that the quality of the DCA contact map prediction for a given input protein increases with the number and diversity of homologs present in the MSA. As the diversity of the input MSA increases, the DCA output becomes increasingly predictive of the true protein contacts. Thus, as described herein, discovering new and diverse homologs is advantageous for co-evolutionary analysis of intra-protein contacts, which in turn, may be used to predict three-dimensional structure.

There are several approaches to generating a list of numerous and diverse homologs of a protein of interest, which can be used to compute co-evolution based, DCA-generated, contact maps as a critical input for predicting protein structure. One of these approaches includes iteratively searching an input sequence against one or more, large curated databases of protein sequences. HHblits (Remmert et al. Nature Methods 2012; 9:173-175) and PSI-BLAST (Altshul et al. Nucleic Acid Res. 1997; 25(17):3389-402) are two of the most sophisticated MSA generation tools available. Both HHBlits and PSI-BLAST are iterative multiple alignment search tools that perform fast and sensitive alignments by searching and comparing compressed MSAs, which take the form of sequence profiles or profile Hidden Markov Models (HMMs), rather than by comparing individual sequences themselves. Also, both tools are iterative, meaning that after performing an initial search for sequences homologous to a target protein sequence, they refine the query sequence profile over additional search rounds using any newly detected homologs from the previous round, adding statistically significant sequence matches to the query profile with each search iteration.

Applicants have discovered, however, that HHblits and PSI-BLAST are limited by the size and scope of the curated protein sequence databases that they search and, therefore, the MSAs they produce depend on the quality of the NCBI non-redundant and Uniprot databases and the pace at which they are updated. Applicants have recognized that this is a limitation for protein prediction software.

### Whole-Genome Metagenomic Sequence Read Archives

In contrast to the large curated protein databases, which contain ˜200 million protein sequences, metagenomic sequencing read archives are among the world's largest databases of biomolecular sequences. For example, the NCBI sequencing read archive (SRA) contains more than 10¹⁶ bp of sequence data and is growing exponentially. Although organizations and tools such as MGnify assemble whole-genome metagenomic datasets from read archives into contigs/whole genomes, annotate predicted protein-coding sequences, and deposit those annotated sequences into curated databases, Applicants have noted that there can be a significant time-lag from when raw nucleic acid sequencing reads are deposited in a sequencing read archive to the submission to a curated database of the protein sequences predicted to be encoded by the genomes represented within, and some raw sequencing reads will never be assembled and curated at all (either because an entire dataset is not assembled/curated, or because some reads within an assembled dataset cannot be placed into sufficiently large contigs).

Although the SRA represents the richest, most up-to-date collection of the world's known genomic/metagenomic sequences, the publicly-available whole-genome metagenomic fraction of the archive includes well over 100,000 individual SRA “runs”, each of which contains unassembled, unannotated sequencing reads from an individual sequencing experiment run. As of 2019, the publicly-available whole-genome metagenomic fraction of the SRA contains ˜2×10¹² reads across >110,000 runs. In this format, the SRA cannot be directly searched by the typical MSA generation tools such as HHBlits and PSI-BLAST. One computational approach, “searchsra” (searchsra.org) can be used to search a fixed sample of nucleic acid sequencing reads from each of the totality of runs in the whole-genome metagenomic fraction of the SRA for nucleic acid sequences homologous (on the nucleic acid or protein level) to a search query.

The SRA, despite its massive size and utility for protein structure prediction, still contains only a tiny fraction of the total number of protein sequences that exist on Earth. Applicants have recognized that there remains an opportunity to mine additional protein-coding sequences directly from new, physical DNA samples that have yet to be sequenced and deposited in any form to a sequence database. However, standard DNA sequencing efforts to mine homologs from diverse DNA samples are unlikely to be the solution, as next-generation sequencing (NGS) technologies permit massively parallel sequencing of DNA, but generate a finite number of reads per sequencing run. While abundant sequences in a given sample are readily detected with high confidence by modern NGS methods, Applicants have appreciated that rare sequences of interest, such as sequences coding for proteins homologous to a protein of interest, may not be sequenced deeply enough, even after multiple runs, to be detectable.

Target Enrichment

Target enrichment sequencing is one approach that can allow for confident base-calling for rare sequences. By enriching a complex sample for a specific gene or region of interest prior to sequencing, a researcher may largely eliminate off-target sequences and thereby only dedicate sequencing reads to genomic regions of interest. Applicants have appreciated that target enrichment can therefore enable the same number of reads to be devoted to a rare region/gene of interest as would require many standard sequencing runs on non-enriched samples, resulting in time and cost savings for homolog discovery.

There are several approaches that enable target enrichment sequencing. The simplest approach is to pre-enrich genomic regions of interest from a complex sample by amplification prior to sequencing, known as amplicon-seq (using, e.g., ILLUMINA® next generation sequencing (NGS) platforms). Primers designed to bind to a target nucleic acid sequence may be used to amplify homologous sequences from a complex mixture, where the nucleic acid sequence between the primer binding sites can diverge from known target-like sequences. However, as Applicants have appreciated, most amplification strategies are not tolerant of mismatches in the primer binding regions themselves. Therefore, amplicon-sequencing is somewhat limited in its ability to enrich homologs that are highly divergent in the primer binding regions. Amplification of full-length homologous genes is therefore especially problematic, as the terminal and flanking regions of genes are unlikely to be well-conserved. Furthermore, exponential amplification approaches can be challenging for nucleic acid targets that are present in very low abundance, since any low abundance nucleic acid not amplified in the first few rounds of amplification are unlikely to be detected at the completion of the reaction. Furthermore, amplification is difficult to multiplex and introduces sequencing errors that can complicate the identification of enriched variants that are truly sequence-divergent from the known target sequence(s).

Alternatively, target enrichment can be performed by nucleic acid hybridization capture. Because similar protein sequences are encoded by similar nucleic acids, and because similar nucleic acids have greater hybridization binding energy than dissimilar nucleic acids due to base pair complementarity, one can use nucleic acid binding assays to isolate nucleic acids from a complex mixture that resemble a given target sequence. There are a number of methods for nucleic acid hybridization capture by target sequence “probes,” including hybridization of complex mixtures to microarrays and to long single-stranded biotinylated oligonucleotide probes, immobilized on magnetic streptavidin beads. What is common to all of these strategies is that after an incubation period during which targets hybridize to the probes, repeated washes remove unbound, off-target sequences, while enriched homologous targets are retained on the immobilized probes. These hybridization-based approaches are more tolerant of mismatches than amplification based enrichment and avoid amplification bias, but they do select for sequences that have low rates of dissociation; if a candidate target dissociates from an immobilized probe during washing, it is removed from the reaction and can no longer be enriched, resulting in the discovery of only those homologs that rarely dissociate from the probes.

There is another hybridization-based technique, known as SCODAphoresis, that may be used to pre-enrich a sample for rare nucleic acids, making the subsequent sequence analysis of those nucleic acids far more effective. SCODAphoresis involves (i) loading a nucleic acid sample on a separation medium containing an immobilized probe, (ii) enriching the sample for nucleic acids complementary to the immobilized probe by applying a time-varying driving field and time-varying mobility field to the separation medium, and (iii) characterizing the enriched nucleic acid in the sample, including by sequencing. See, e.g., U.S. Pat. Nos. 9,512,477 and 9,534,304, incorporated herein by reference.

To date, for all of these approaches, target-enrichment sequencing has mostly been applied for the purpose of enriching clinical and/or human genomic samples for genes or panels of genes of interest. Herein, pre-enrichment allows for the devotion of fewer sequencing reads to a sample containing a single gene or collection of genes (e.g., cancer panel, or human exome) while maintaining high coverage. This results in cost and time savings. High read coverage is often used to allow for better gene variant determination, especially for the purposes of characterizing rare, disease causing genetic variants. Target enrichment has found ready application for single nucleotide polymorphisms (SNPs), insertion/deletion (indel) deletion, copy number variation (CNV) detection, and structural variation detection.

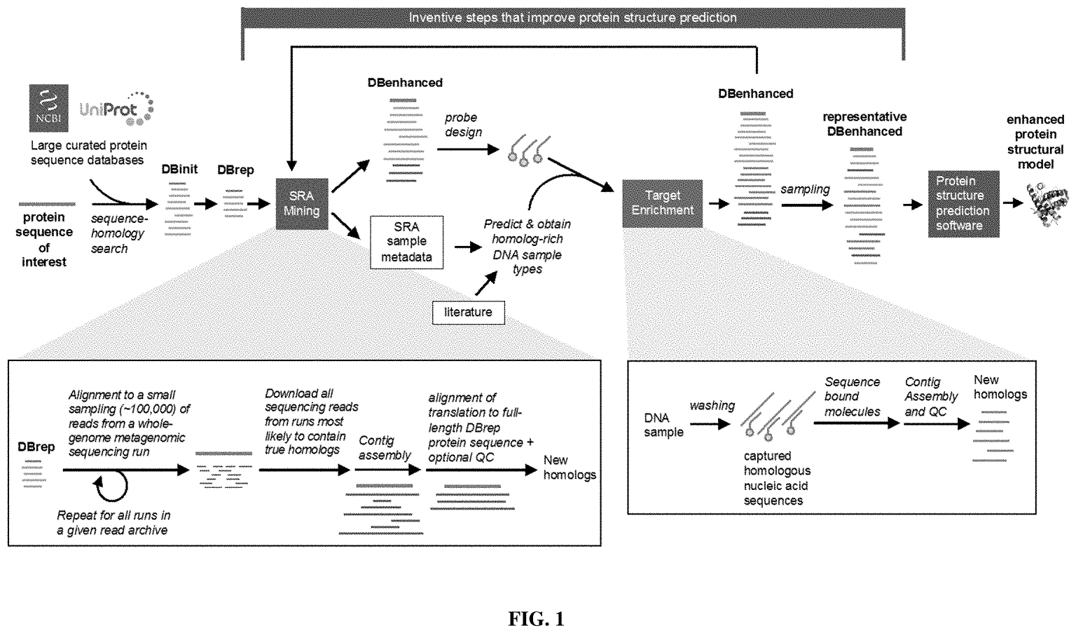

The present disclosure provides, in some embodiments, methods that use hybridization capture-based target enrichment for the intentional mining of highly divergent homologs (rather than more closely-related/similar homologs) for a known protein to enhance structural prediction. FIG. 1 is a flow diagram of the steps of an illustrative process for discovering protein homologs, such as divergent protein homologs, which may include in silico homolog mining from metagenomic sequencing read databases and target enrichment. The methods provided herein, in some embodiments, are used for building an improved MSA for protein structure prediction that is larger and more diverse than MSAs compiled to date. This improved MSA can be used to generate higher quality DCA outputs, for example, which can be used in turn to train higher quality protein structure prediction models and execute higher quality de novo protein structure prediction.

In some embodiments, a method of the present disclosure comprises at least one (e.g., 1, 2, 3, 4, 5, 6, 7, 8, 9, 10, 11, 12, or 13) of the following steps:
1. generating an initial homolog list for protein/protein family of interest by a sequence-homology search (pairwise or profile HMM-based; pre-computed or not) of one or more protein sequence databases;
2. from the initial list, generating a representative database (DBrep) of homologs related to the protein of interest (includes optional quality-control steps);
3. aligning the DBrep to a relatively small sampling of reads/read-pairs (e.g., 100,000) from every “whole-genome metagenomic” run in the SRA using searchsra.org;
4. ranking datasets prior to downloading to determine which are most likely to contain the most true homologs; ranking features can include (before/after false-positive removal):
   a. number of reads/read pairs in the 100,000-read sample giving an alignment probability value with DBrep above a certain threshold (“hit reads”);
   b. diversity of hit reads from the 100,000-read sample;
   c. total number of reads in the run;
   d. average length of reads;
   e. average length of hit read alignments;
   f. sequencing platform used; and
   g. Rread format (eg. paired or un-paired);
5. retrieving all reads from each “hit” (highly-ranked) SRA run;
6. optionally performing quality control steps to clean up unassembled reads from each “hit”SRA run;
7. aligning the DBrep protein list (with e.g., DIAMOND (Buchfink et al., Nat Methods 2015; 12: 59-60) or AC-DIAMOND or profile (with e.g., HMMSEARCH (Eddy et al. PLoS Computational Biology 2011; 7(10):e1002195)) to all nucleic acid reads/read-pairs or translated reads/read-pairs from every “hit” SRA run;
8. for each “hit” SRA run, assembling all full-length nucleic acid reads/read-pairs aligning to DBrep into contigs, using a fast assembler appropriate for the run's read format (paired/unpaired) and length (e.g., IDBA-UD (Peng et al., Bioinformatics 2012; 28(11): 1420-1428) for short reads);
9. translating open reading frames (ORFs) (e.g., all six possible ORFs) from assembled contigs to generate candidate protein homologs;
10. optionally performing quality control steps to validate candidate protein homologs as true homologs;
11. adding new homologs to the initial homolog list;
12. generating a new representative multiple sequence alignment (MSA) that has optimal balance of size and sequence diversity for DCA; and
13. performing feature extraction using the new MSC for co-evolution based protein structure prediction model.

It is envisioned that at least some of these steps can be implemented by a processor such as that included in a computer (e.g., a general purpose computer).

### An in Silico Multiple Sequence Alignment Method for Use in Co-Evolution-Based Protein Structure Prediction

There are trillions of sequencing reads/read pairs in the “whole-genome metagenomic” fraction of the NCBI Sequencing Read Archive (SRA) and additional sequencing reads in other metagenomic read archives (e.g. MG-RAST), and Applicant have appreciated that only a fraction of which have been assembled into contigs, annotated, undergone coding sequence translation and deposited into the large, curated NCBI and/or uniprot protein databases (200+ million protein sequences). In particular, metagenomic samples may include DNA from a multitude of organisms, spanning multiple kingdoms of life, including those that have never been previously identified, cultured or sequenced and thus contain highly diverse sequencing reads. Applicants have therefore recognized that metagenomic datasets represent a trove of additional protein sequences, from which homologs of a protein of interest may be identified.

A general illustrative method for in silico mining for new protein homologs includes the following steps.
1. Identifying a protein of interest for which a 3D structure is to be predicted.
2. Building an initial protein homolog sequence list, DBinit, for the protein of interest. This can be achieved by a number of means, including, for example:
   a. Searching protein family databases (e.g., InterPro, Pfam, CDD) for all proteins containing a given protein domain (architecture).
   b. Searching the NCBI non-redundant and/or uniprot protein sequence databases using pairwise (eg. BLAST, DIAMOND, AC-DIAMOND, PSI-BLAST), or profile HMM-based (eg. HHblits, JACKHMMER) alignment.
3. Optional: Assessing the completeness of the initial homolog list by downloading the entire NCBI non-redundant (nr) protein reference database and using it as a query against the DBinit initial database using DIAMOND, a fast and sensitive protein alignment tool adapted for large query sets, to search it for additional hits.
   a. To eliminate false-positive hits from this NCBI non-redundant search, the “Blast Score Ratio (BSR)” normalization method as described by Rasko et al. BMC Bioinformatics (2005) can be implemented, where the BLAST score for each non-redundant query hit against DBinit is normalized by its maximum possible score (a self-hit).
   b. Appending all true positives to DBinit.
4. Generating a representative reference database (DBrep) for all members of the protein family of interest by eliminating the presence of multiple sequences in DBinit that are very close in amino acid sequence space to each other. One non-limiting approach for doing this is to cluster DBinit by amino acid percent identity. For example, generate DBrep by clustering DBinit at, e.g., 90% using UCLUST.
5. Screening the SRA with the DBrep query using the public searchsra.org service to sample 100,000 reads from each of the “whole-genome metagenomic” runs in the SRA, likely revealing read hits over multiple individual SRA runs. Note that 100,000 reads is typically ˜1% of the complete dataset for any given SRA run, and thus represents a small fraction of the total reads.
6. Ranking datasets prior to downloading to determine which are most likely to contain the most true homologs. Ranking features can include (before/after false-positive removal):
   a. number of reads/read pairs in the 100,000-read sample giving an alignment probability value with DBrep above a certain threshold (“hit reads”);
   b. diversity of hit reads from the 100,000-read sample;
   c. totaling number of reads in the run;
   d. averaging length of reads;
   e. averaging length of hit read alignments;
   f. sequencing platform used; and
   g. reading format (eg. paired or un-paired).
7. Downloading the complete SRA run (all reads, not just a 100,000-read sampling) for any SRA runs that had positive hits in the 100,000-read sample OR a subset of those runs, for example, as triaged by the above ranking system, such that there is a minimum threshold rank to warrant downloading. Full SRA datasets are needed to search the entirety of the runs for additional reads that align to DBrep, to obtain high enough coverage of those genomic regions to be able to stitch shorter reads together into contigs that cover the full length of the protein of interest. Downloading can be performed using a number of approaches, including:
   a. manually downloading of individual SRA runs of interest;
   b. using commercial Aspera software, optimizing for efficient file transfer; and
   c. implementing a cloud transfer protocol to access SRA data in AWS (Amazon Web Service) or GCP (Google Cloud Computing) servers. This would allow for rapid, automatic execution of the pipeline and is the most robust option.
8. For each of the downloaded SRA run datasets, using an alignment tool to align all reads to the DBrep reference database. Multiple alignment tools could be used, including DIAMOND and HMMSEARCH (which requires translation first).
   a. Optional: Prior to contig assembly, aggregate reads from runs with the same sample origin to improve coverage.
9. For each dataset, assembling all hit reads into contigs. Multiple assemblers could be used, including:
   a. iterative de Bruijn Graph Assembler optimized for metagenomic data (IDBA-UD);
   b. a collection of different assemblers to be used across different SRA runs, where a strategy is used to identify the most optimal assembler for a given SRA run according to its unique read characteristics (e.g., read length, read format, coverage, etc); and/or
   c. de novo or reference-guided assemblers.
   d. Optional: Prior to assembly, false-positive hit read removal may be performed.
10. Open Reading Frames (ORFs) resulting in protein sequences greater than a cutoff fraction (e.g., 0.5-1.0, e.g., 0.7) of the length of the average DBrep protein member are then translated from these contigs in (e.g., all six (6)) reading-frames.
11. Translated ORFs in (e.g., all six (6)) reading-frames can be directly aligned (protein-protein) to DBrep to identify protein sequences aligning over a cutoff fraction (e.g., 0.5-1.0, e.g., 0.7) of the length of a DBrep member sequence.
12. Optional: Additional quality control steps may be performed, including of the following steps:
   a. detecting and remove artificial chimeras;
   b. aligning putative new homologs to all known protein sequences in a protein sequence database (e.g. NCBI nr) and the initial full database (DBinit); and
   c. if alignment to DBinit is better than to any non-DBinit member from NCBI nr, then putative homolog is considered a true homolog; and
13. Adding new homolog protein sequences to DBinit, generating an enhanced homolog listing, or DBenhanced.

It also is envisioned that at least some of these steps can be implemented by a processor such as that included in a computer (e.g., a general purpose computer).

### A Target Enrichment Sequencing Method for Enhancing a Multiple Sequence Alignment for Use in Co-Evolution Based Protein Structure Prediction

Protein coding DNA sequences from only a small percentage of life on Earth have been extracted, sequenced, annotated, and deposited into curated protein sequence databases. Target enrichment directly from previously uncharacterized DNA samples, including metagenomic samples, for the identification of new protein homologs is therefore especially advantageous for expanding the size and diversity of the list of known homologs of a protein of interest.

In some embodiments, a method of the present disclosure comprises the following steps:
1. generating an initial MSA for protein/protein family of interest by a sequence-homology search (pairwise or profile HMM-based; pre-computed or not) of one or more protein sequence databases;
2. from the initial MSA, designing one or more probes (e.g., nucleic acid, e.g., DNA, probes) that can hybridize to nucleic acid sequences that broadly represent the protein homolog family of interest;
3. immobilizing probes on a solid substrate, which could include a separation medium;
4. contacting probes with physical, complex DNA sample;
5. enriching homologs from non-homologs by selectively removing DNA unbound to the probes;
6. releasing bound homologs from the probes and sequence the DNA;
7. performing quality control steps to clean up sequencing reads;
8. aligning reads to the initial MSA used for probe design and if reads are shorter than the length of the full-length target sequence, assemble reads that positively align into contigs;
9. translating ORFs from aligned contigs to generate candidate protein homologs;
10. performing quality control steps to validate candidate protein homologs as true homologs;
11. adding new homologs to the MSA;
12. generate subset of the total MSA that has optimal balance of size and sequence diversity for DCA; and
13. performing feature extraction for co-evolution based protein structure prediction model.

One skilled in the art understands that there are multiple target enrichment strategies that may be employed. SCODAphoresis, for example, may be used for mining homologs from physical samples. In some embodiments, SCODAphoresis is used to purify divergent homologs from whole samples, where probes and target enrichment conditions are designed to enrich as many sequence variants as possible with relaxed stringency.

It also is envisioned that at least some of these steps can be implemented by a processor such as that included in a computer (e.g., a general purpose computer).

Probe Design

In some embodiments, designing a probe comprises at least one (e.g., 1, 2, 3, 4, 5, 6, 7, or 8) of the following steps.
1. Identifying a protein of interest for which a 3D structure is to be predicted.
2. Building an initial protein homolog sequence list, DBinit, for the protein of interest. This can be achieved by a number of means, including:
   a. searching protein family databases (eg. InterPro, Pfam, CDD) for all proteins containing a given protein domain (architecture); and
   b. searching the NCBI non-redundant and/or uniprot protein sequence databases using pairwise (eg. BLAST, DIAMOND, AC-DIAMOND, PSI-BLAST), or profile HMM-based (eg. HHblits, JACKHMMER) alignment.
3. Optional: assessing the completeness of the initial homolog list by downloading the entire NCBI non-redundant protein reference database and using it as a query against the DBinit initial database using DIAMOND, a fast and sensitive protein alignment tool adapted for large query sets, to search it for additional hits.
   a. To eliminate false-positive hits from this NCBI non-redundant search, implementing the “Blast Score Ratio (BSR)” normalization method as described by Rasko et al (2005), where the BLAST score for each non-redundant query hit against DBinit is normalized by its maximum possible score (a self-hit);
   b. Appending all true positives to DBinit.
4. Retrieving associated nucleic acid sequences associated with each protein record.
5. Generating an MSA for all members of the protein family of interest at the nucleotide level.
6. Generating a representative MSA (MSAref) by eliminating the presence of multiple sequences in MSA initial that are very close in sequence space to each other.
   a. One approach (among others) for doing this is to cluster MSA initial by percent identity.

For example, generate MSAref by clustering MSA initial at 90% using UCLUST.
7. From MSAref, calculating the associated position-specific weight matrix (PWM). The PWM calculates both total information content and the weighted probability of finding any given nucleotide base for each individual position in the alignment.
8. Designing an optimal set of “probe” sequences most likely to hybridize to newly found homologs by:
   a. scanning through a sliding window of the MSA for different possible probe lengths;
   b. for each candidate probe (window of the MSA), calculating a probe score, comprised of the following metrics:
      i. mean information content (IC) from PWM;
      ii. longest sub-stretch of high IC bases;
      iii. percentage of low IC (degenerate) bases;
      iv. GC content (weighted by PWM);
      v. self-dimerization energy of consensus sequence; and/or
      vi. hairpin formation energy of consensus sequence;
   c. ranking probes by score and remove overlapping probes according to probe score, keeping the set of the most highly ranked, non-overlapping probes; and
   d. determining the optimal set of the most highly ranked, non-overlapping probes, with the lowest hetero-dimerization potential.
      i. One approach is to begin with the most highly ranked probe and calculate the hetero-dimerization potential for adding the 2^{nd} most highly ranked probe. If this passes an energy threshold, then add the 3^{rd} most highly ranked probe and repeat. If the 2^{nd} most highly ranked probe does not pass, move onto the 3^{rd} most highly ranked probe. Continue until the energy threshold can no longer be met.
         Features of designed probes that are important for homolog mining:
   a. Probes can include non-standard nucleotide bases.
      i. Probes can include mixed/degenerate bases to increase the diversity of nucleic acid sequences that can be strongly bound/hybridized.
      ii. Probes can include locked nucleic acids and peptide nucleic acids to increase the melting temperature of a probe-target hybridization event.
      iii. Probes can include “universal” bases that base-pairing to multiple nucleotide bases, including 5′-nitroindoles and deoxyInosine bases, to increase the diversity of nucleic acids that can be strongly bound/hybridized.
   b. Optional: Simultaneously immobilize multiple probes for multiplexed target capture.
      i. Non-overlapping probes that tile the length of a target sequence can be immobilized in a single gel to increase the diversity of nucleic acid enrichment—so long as a target hybridizes to one probe it can be enriched, even if its sequence is divergent at the other probe sites.
      ii. Simultaneously enrich for multiple targets.
   c. Probes can hybridize nucleic acid targets anywhere along the sequence—in the middle or at the ends (unlike PCR based enrichment that requires the binding of two probes at opposite ends of a target molecule).
      i. Longer probes increase the diversity of nucleic acid enrichment by permitting hybridization to molecules that align at a minimum to a subsequence within the long probe.

It also is envisioned that at least some of these steps can be implemented by a processor such as that included in a computer (e.g., a general purpose computer).

Method for Fragmenting DNA Sample

The following is one example of a method for fragmenting a DNA sample.
1. Obtain whole samples from which new homologs are to be enriched. The following are features of nucleic acid containing samples that are important for target enrichment.
   a. Mobile samples can be complex, containing mixtures of nucleic acids with varying sequence homology to the probe set and non-nucleic acid molecules.
      i. Individual nucleic variants with high homology to the nucleic probe set can be extremely rare in the original sample.
      ii. Enrichment can be performed with metagenomic samples extracted from the environment that contain unknown mixtures of molecules, some of which have never previously been characterized.
      iii. Enrichment can be performed with samples isolated from one or more known organisms.
   b. Enriched nucleic acids can be linear or circular DNA molecules.
   c. Enriched nucleic acids can be single stranded or intact duplex DNA molecules.
      1. Can be fragmented by transposase.
      2. Can be fragmented by mechanical shearing.
      3. For example, can be fragmented to <3 kb for use with acrydite modified oligonucleotides immobilized in an acrylamide gel.
   d. Enrichment can be visualized and quantified by the incorporation of fluorescent dyes into the nucleic acid molecules undergoing enrichment.
2. Extract DNA from the sample using the appropriate method according to the sample type.
3. Optional: Samples that contain high molecular weight DNA can be fragmented prior to target enrichment. For SCODAphoresis, this would mean generating 1-3 kb fragments to facilitate electrophoretic mobility of the sample in the separation medium. Fragments may be generated by:
   a. physical DNA fragmentation (e.g. sonication, shearing);
   b. chemical fragmentation; and/or
   c. enzymatic fragmentation (e.g., nuclease, transposase treatment).
4. Ligate adapter sequences to the 3′ and 5′ ends of the fragmented DNA molecules to be used as PCR primer handles downstream.
5. In one implementation, fragmentation and adapter ligation are combined in a single transposase mediated step:
   a. assemble transposomes consisting of annealed adapter oligos and MBP-tagged Tn5 transposase enzyme (transposomes may be used fresh, or stored frozen);
   b. prepare reaction with transposomes and DNA at 10:1 Tn5:DNA mass ratio; incubate at 55° C. for 80 minutes;
   c. stop fragmentation and adapter addition (aka “tagmentation”) reaction by adding 0.2% SDS and incubating at 55° C. for 10 min;
   d. clean up DNA reaction with size-selection using SPRI (e.g., AMPure) beads
6. Optional: To generate more adapter-appended, fragmented DNA, perform PCR amplification. To minimize PCR bias, chimeric product generation, and other errors during amplification:
   a. use 0.1 ng/uL DNA template (final concentration in the amplification reaction); and/or
   b. amplify for 12 cycles.

It also is envisioned that at least some of these steps can be implemented by a processor such as that included in a computer (e.g., a general purpose computer).

Method for Targeted Enrichment

The following is one illustrative example of a target enrichment process.
1. Flow complex DNA sample over immobilized probes in hybridization buffer.
2. Remove weakly or non-specifically hybridized “off-target” DNA molecules by repeated washing.
3. Release tightly, specifically hybridized “target” DNA molecules from the immobilized probes.

In some embodiments, SCODAphoresis is used for target enrichment of divergent homologs from a DNA sample. An instrument that can perform SCODAphoresis (i) contains multiple electrodes for generating dynamic electric fields (ii) Contains one or more temperature controllers for the uniform or non-uniform generation of temperature gradients in the electrophoresing gel (iii) incorporates sample inlet ports, enriched sample recovery port, outlet ports for highly mobile sequences.

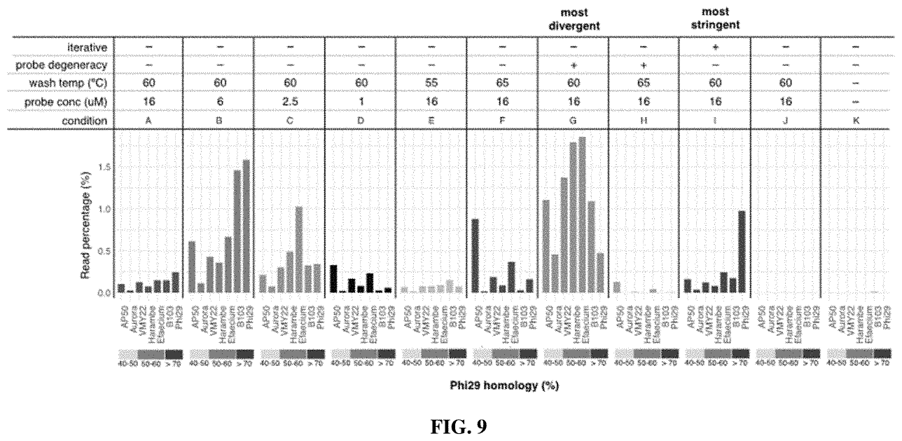

SCODAphoresis, in some embodiments, may include the following steps:
1. The separation of nucleic acid variants is achieved by repeated on/off binding interactions between nucleic acids and immobilized probes that results in a differential mobility for each individual nucleic acid variant.
2. The mobility of nucleic acids is driven by an electric field, resulting in electrophoresis of nucleic acid variants through gel-immobilized probes.
3. A user can remove higher mobility (less tightly bound) sequences by electrophoresing them away and thereby enrich the remaining (more tightly bound) sequences.
4. A nucleic acid can still be low mobility in the gel, but contain multiple mismatches to the probe—non perfect sequence complementarity.
5. Control over the stringency of the separation is tuned by temperature, the number of enrichment iterations, probe concentration, and probe design. See FIG. 9, which suggests that through interaction of all of these parameters, the stringency of enrichment of a sample can be tuned—where high stringency target enrichment purifies nucleic acids most homologous to the original target (Phi29) and more relaxed target enrichment purifies even divergent (40-50% homology) nucleic acids.

It also is envisioned that at least some of these steps can be implemented by a processor such as that included in a computer (e.g., a general purpose computer).

### An Iterative Homolog Discovery Method Including Synergistic in Silico and Physical Assays

In silico homolog discovery enables metagenomic sequencing reads collected from locations across Earth's biosphere to be screened broadly (but shallowly, since sequence reads were not pre-enriched) for homologs of a given target sequence. In the process, metagenomic archive mining gathers two useful pieces of information (1) an expanded set of homologs for probe design, and (2) from the sequencing read metadata, identification of which ecosystems or organisms were the richest in homologs, suggesting where to sample in the future. Hybridization capture target enrichment can then be applied to newly collected physical samples likely to be enriched for the protein family of interest, and then enrich it from homologous sequences thousands-millions times more, much like an oil-drill is applied after global screens. Once target enrichment reveals additional homologs, one can return to in silico homolog mining and search for further homologs from the expanded definition of the homolog family Algorithms that work only on large curated protein sequence databases (such as PSI-BLAST and HHblits) use such an iterative strategy for extra-sensitive homology searches. The present disclosure provides, in some embodiments, an iterative strategy between in silico broad sequencing-read archive searches and physical, narrow target enrichment searches, creating a synergistic cycle between the two.

In some embodiments, a method of the present disclosure comprises the following steps:
1. generating an initial homolog list for protein/protein family of interest by a sequence-homology search (pairwise or profile HMM-based; pre-computed or not) of one or more protein sequence databases;
2. metagenomic sequence read homolog mining (see Example 1) broadly screens submitted metagenomic sequencing reads for new homologs;
3. based on the lengthened MSA (includes new homologs identified by in silico mining), designing “probes” for target nucleic acids;
4. downloading metadata for metagenomic samples with positive homolog identification to reveal the ideal sample collection type and location for the target protein family;
5. obtaining a physical DNA sample predicted to be rich with putative homologs;
6. performing hybridization-capture target enrichment with designed probes and chosen DNA sample (see above);
7. from target enrichment sequencing data, identifying new homologs;
8. generating lengthened MSA; and
9. with lengthened MSA, repeating steps 2-8 (repeating iteratively).

It also is envisioned that at least some of these steps can be implemented by a processor such as that included in a computer (e.g., a general purpose computer).

### Computer Implementation

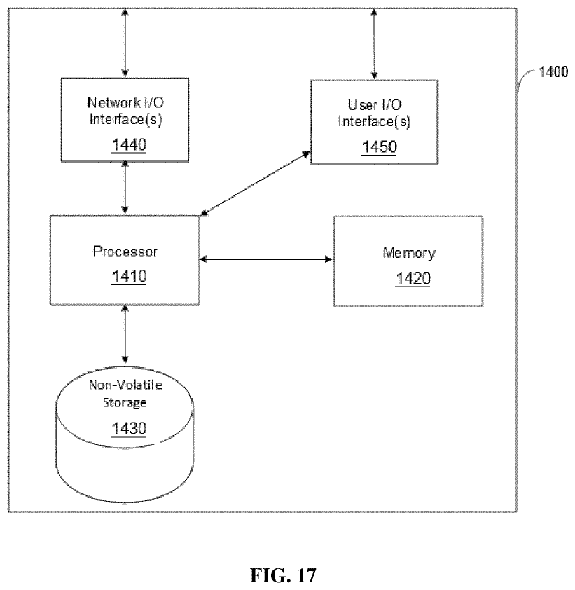

An illustrative implementation of a computer system 1400 that may be used in connection with any of the embodiments of the technology described herein is shown in FIG. 17. The computer system 1400 includes one or more processors 1410 and one or more articles of manufacture that comprise non-transitory computer-readable storage media (e.g., memory 1420 and one or more non-volatile storage media 1430). The processor 1410 may control writing data to and reading data from the memory 1420 and the non-volatile storage device 1430 in any suitable manner, as the aspects of the technology described herein are not limited in this respect. To perform any of the functionality described herein, the processor 1410 may execute one or more processor-executable instructions stored in one or more non-transitory computer-readable storage media (e.g., the memory 1420), which may serve as non-transitory computer-readable storage media storing processor-executable instructions for execution by the processor 1410.

Computing device 1400 may also include a network input/output (I/O) interface 1440 via which the computing device may communicate with other computing devices (e.g., over a network), and may also include one or more user I/O interfaces 1450, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. The software can be coded in any suitable programming language and when implemented by a processor cause that processor to perform at least some of the steps listed in the methods described. Some of the algorithms coded in software may be artificial intelligence machine learning algorithms, trained on an initial set of data, and learn and improve as more data is fed into the system.

It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

### Additional Embodiments

Additional embodiments of the present disclosure are encompassed by the following numbered paragraphs.

1. A method of in silico mining for new homologs of a protein of interest, the method comprising:

producing an initial protein homolog sequence database (DBinit) for the protein of interest;

generating a representative reference database (DBrep) of putative protein homolog sequences by eliminating multiple sequences in the DBinit that share at least 75% identity;

screening a metagenomic sequencing read archive, optionally a sequencing read archive, using the DBrep as a query to identify datasets of sequencing reads, and optionally ranking the datasets to determine which are most likely to contain the highest number of true homologs;

aligning the DBrep to the sequencing reads, optionally all sequencing reads, from a given metagenomic dataset;

assembling the aligned sequencing reads into contigs;

translating open reading frames (ORFs) of the contigs into protein sequences having greater than a cutoff fraction of the length of the average DBrep protein sequence;

aligning the translated protein sequences with the DBrep protein sequences and identifying new putative protein homolog sequences, and optionally adding the new putative protein homolog sequences to the DBinit to produce an enhanced protein homolog sequence database (DBenhanced).

2. The method of paragraph 1, wherein the producing a protein homolog sequence database includes searching protein family databases for proteins containing a conserved protein domain.

3. The method of paragraph 1, wherein the producing a protein homolog sequence database includes searching protein sequence databases using pairwise or hidden Markov model (HMM)-based alignment.

4. The method of any one of the preceding paragraphs, further comprising assessing completeness of the DBinit by aligning a known non-redundant protein reference database and the DBinit, optionally using a protein alignment tool adapted for large query sets, and searching for additional homologs of the protein of interest.

5. The method of any one of the preceding paragraphs, wherein the DBprep is generated by clustering the DBinit at 90% using a clustering algorithm.

6. The method of any one of the preceding paragraphs, wherein the aligning the DBrep to sequencing reads of each of the SRA datasets comprises aligning the DBrep to a sampling of reads/read-pairs from every whole-genome metagenomic run in the SRA, optionally wherein the sampling size is about 100,000 reads.

7. The method of any one of the preceding paragraphs, further comprising quality control steps to remove unassembled reads from the metagenomic datasets.

8. The method of any one of the preceding paragraphs, wherein the translating comprises translating six ORFs of the contigs.

9. The method of any one of the preceding paragraphs, further comprising quality control steps to validate the putative protein homolog sequences as true protein homolog sequences, which are then optionally added to the DBenhanced.

10. The method of any one of the preceding paragraphs, further comprising target protein enrichment.

11. The method of any one of the preceding paragraphs, further comprising generating a representative multiple sequence alignment (MSA) based on the DBenhanced.

12. A target enrichment method comprising:

providing a list of putative protein homolog sequences of a protein of interest from a multiple sequence alignment (MSA) of sequences homologous to the protein of interest;

contacting a sample comprising DNA with probes to produce probes bound to DNA, wherein the probes are designed to hybridize, optionally with low stringency, to the nucleotide sequences of the putative protein homolog sequences, and wherein the probes are immobilized on a substrate that optionally includes a separation medium;

optionally selectively removing from the substrate probes that are not bound to DNA;

sequencing the DNA bound to the probes to produce sequencing reads;

aligning the sequencing reads to the MSA and assembling contigs from any sequencing reads that are shorter than the full-length sequence of the protein;

translating open reading frames (ORFs) from the contigs to generate new putative protein homolog sequences, and optionally validating the new putative protein homolog sequences as true protein homolog sequences; and

optionally adding the new putative protein homolog sequences to the MSA to produce an enriched MSA.

13. The method of any one of the preceding paragraphs, further comprising executing on the MSA an algorithm for deducing direct correlation, optionally wherein the algorithm is a Direct Coupling Analysis (DCA) algorithm.

14. The method of any one of the preceding paragraphs, further comprising performing feature extraction using the enriched MSA for a co-evolution-based protein structure prediction model.

15. An iterative homolog discovery method comprising:

(a) performing the method of any one of paragraphs 1-11 to produce an enhanced multiple sequence alignment (MSA);

(b) performing the target enrichment method of any one of paragraphs 12-14 to identify new putative protein homolog sequences, wherein the DNA sample has been identified using metadata for metagenomic SRA samples with positive homolog identification;

(c) adding the new putative protein homolog sequences to the enhanced MSA; and

(d) optionally repeating the steps (a)-(c) iteratively.

16. A computer readable medium on which is stored a computer program which, when implemented by a computer processor, causes the processor to:

produce an initial protein homolog sequence database (DBinit) for the protein of interest;

generate a representative reference database (DBrep) of putative protein homolog sequences by eliminating multiple sequences in the BDinit that share at least 75% identity;

screen the sequencing read archive (SRA) using the DBrep as a query to identity datasets of sequencing reads, and optionally rank the datasets to determine which are most likely to contain the highest number of true homologs.

17. The computer readable medium of paragraph 16, wherein the computer program further causes the processor to:

align the DBrep to sequencing reads of the SRA datasets to identify hit reads;

assemble hit reads into contigs;

translate open reading frames (ORFs) of the contigs into protein sequences having greater than a cutoff fraction of the length of the average DBrep protein sequence;

align the translated protein sequences with the DBrep protein sequences and identifying new putative protein homolog sequences, and optionally add the new putative protein homolog sequences to the DBinit to produce an enhanced protein homolog sequence database (DBenhanced).

18. A computer readable medium on which is stored a computer program which, when implemented by a computer processor, causes the processor to:

align sequencing reads to a multiple sequence alignment (MSA) and assembling contigs from any sequencing reads that are shorter than a full-length sequence of the protein;

translating open reading frames (ORFs) from the contigs to generate new putative protein homolog sequences; and

add the new putative protein homolog sequences to the MSA to produce an enriched MSA.

19. A computer implemented method of mining for new homologs of a protein of interest, the method comprising:

producing an initial protein homolog sequence database (DBinit) for the protein of interest;

generating a representative reference database (DBrep) of putative protein homolog sequences by eliminating multiple sequences in the DBinit that share at least 75% identity;

screening a metagenomic sequencing read archive using the DBrep as a query to identity datasets of sequencing reads, and optionally ranking the datasets to determine which are most likely to contain the highest number of true homologs;

aligning the DBrep to sequencing reads of the metagenomic datasets;

assembling the aligned sequencing reads into contigs;

translating open reading frames (ORFs) of the contigs into protein sequences having greater than a cutoff fraction of the length of the average DBrep protein sequence;

aligning the translated protein sequences with the DBrep protein sequences and identifying new putative protein homolog sequences, and optionally adding the new putative protein homolog sequences to the DBinit to produce an enhanced protein homolog sequence database (DBenhanced).

20. The computer implemented method of paragraph 1, wherein the producing a protein homolog sequence database includes searching protein family databases for proteins containing a conserved protein domain.

21. The computer implemented method of paragraph 19 or 20, wherein the producing a protein homolog sequence database includes searching protein sequence databases using pairwise or hidden Markov model (HMM)-based alignment.

22. The computer implemented method of any one of the preceding paragraphs, further comprising assessing completeness of the DBinit by aligning a known non-redundant protein reference database and the DBinit, optionally using a protein alignment tool adapted for large query sets, and searching for additional homologs of the protein of interest.

23. The computer implemented method of any one of the preceding paragraphs, wherein the DBprep is generated by clustering the DBinit at 90% using a clustering algorithm.

24. The computer implemented method of any one of the preceding paragraphs, wherein the aligning the DBrep to sequencing reads of each of the SRA datasets comprises aligning the DBrep to a sampling of reads/read-pairs from every whole-genome metagenomic run in the SRA, optionally wherein the sampling size is about 100,000 reads.

25. The computer implemented method of any one of the preceding paragraphs, further comprising quality control steps to remove unassembled reads from the SRA datasets.

26. The computer implemented method of any one of the preceding paragraphs, wherein the translating comprises translating six ORFs of the contigs.

27. The computer implemented method of any one of the preceding paragraphs, further comprising quality control steps to validate the putative protein homolog sequences as true protein homolog sequences, which are then optionally added to the DBenhanced.

28. The computer implemented method of any one of the preceding paragraphs, further comprising target protein enrichment.

29. The computer implemented method of any one of the preceding paragraphs, further comprising generating a representative multiple sequence alignment (MSA) based on the DBenhanced.

30. A computer implemented iterative homolog discovery method comprising:

(a) performing the method of any one of paragraphs 19-29 to produce an enhanced multiple sequence alignment (MSA);

(b) inputting results new putative protein homolog sequences obtained from the target enrichment method of any one of paragraphs 12-14, wherein the DNA sample has been identified using metadata for metagenomic SRA samples with positive homolog identification;

(c) adding the new putative protein homolog sequences to the enhanced MSA; and (d) optionally repeating the steps (a)-(c) iteratively.

### EXAMPLES

### Example 1. In Silico Mining for New Protein Homologs

The sequencing read archive (SRA) is a partially publicly accessible archive of most of the world's Next-Gen Sequencing (NGS) data, carrying a massive amount of genetic information, including the sequences of naturally-occurring proteins homologous to a protein of interest. Specifically, the set of >110,000 “whole-genome metagenomic” NGS datasets (“runs”) holds the (partial) sequences of >1.5×10¹² randomly-sampled DNA fragments from communities of microbes isolated across the globe from various ecosystems and host organisms (these sequencing “reads” are typically 100-250 bases in length, often coming in pairs constructed from the 2 ends of a fragment, but in rarer cases can extend to several kilobases).

The methods herein apply SRA mining for the purposes of assembling a superior MSA for protein structure prediction. No protein structure prediction software to date uses an MSA building approach that is compatible with raw nucleic acid sequencing read datasets such as those in the SRA. The bigger and more diverse an MSA is, the higher the quality of the DCA that can be performed, the more precise the generated contact map estimation, and the more accurate the 3D structure prediction.

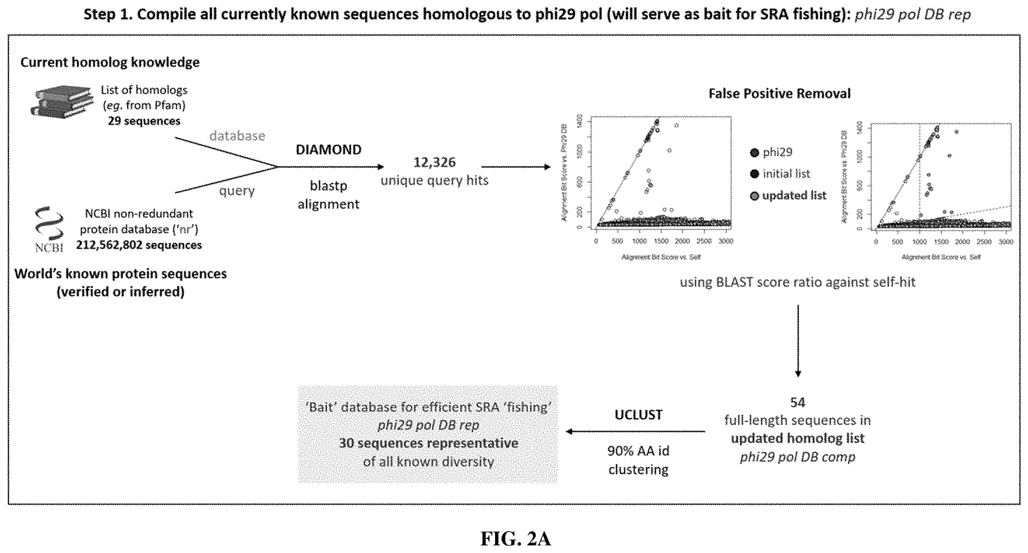

SRA mining was performed to discover as many homologs of the Phi29 DNA polymerase as possible using the following protocol. The results are captured in FIGS. 2A and 2B.

An initial database (DBinit) was composed of 29 unique DNA polymerase sequences known to be homologs of Phi29 DNA polymerase. The completeness of DBinit was assessed by downloading the entire NCBI non-redundant (nr) protein reference database and using it as a query against the DBinit initial database using DIAMOND, a fast and sensitive protein alignment tool adapted for large query sets, to search it for additional hits. There were 12,326 unique query hits against DBinit in the NCBI non-redundant database (default parameters). To eliminate false positive hits, (i) the score of the hit against DBinit and (ii) the maximum possible score (e.g., self-hit) were calculated for each of the 12,326 unique polymerase query hits. Of the 12,326 query hits, 25 Phi29-like sequences were determined to be “real” hits by the Blast Score Ratio. All 25 full-length phi29 DNA polymerase homolog protein sequences were appended to the DBinit, increasing its size to a total of 54 unique sequences.

The 54 phi29-like DNA polymerase sequences in DBinit were then clustered at 90% identity using UCLUST to generate a reference database (DBrep) consisting of 30 representative Phi29-like DNA polymerase protein sequences. Searchsra with DBrep was then run as the database using the public searchsra.org service to sample 100,000 reads/read-pairs from each of the ˜107,000 “whole-genome metagenomic” runs in the SRA processed by searchsra.org (as of October 2019), revealing 369,913 read hits over 25,440 individual SRA runs (datasets). 10 of the SRA run datasets that returned the most read hits from the 100,000-read sampling were manually downloaded, formatted and cleaned. Of these 10 datasets, the 7 datasets containing paired-end reads (better for contig assembly) were selected for further analysis. For each of the 7 SRA run datasets, all reads were searched against the DBrep database and the same ultra-fast DNA-protein aligner as searchsra.org: DIAMOND. For each dataset, full-length hit reads were assembled de novo into contigs using an Iterative de Bruijn Graph Assembler optimized for metagenomic data (IDBA-UD).

Open Reading Frames (ORFS) resulting in protein sequences >70% the length of the average Phi 29 pol DB member were then translated from these contigs in all 6 reading frames. The translated ORFs in all 6 frames were aligned directly to DBrep to find protein sequences (putative new homologs) aligning over 70% of the length of a DBrep member sequence. A final stringency step (see Step 12 above) was then performed to ensure that detected homologs were closer to a member of the complete DB (DBinit) than to any other of the world's known proteins, revealing 13 brand-new, diverse phi29 DNA polymerase protein homologs. New homologs were added to DBinit, generating an enhanced homolog listing, or DBenhanced.

### Example 2. Target Enrichment for New Protein Homologs

Target enrichment sequencing involves the pre-treatment of a DNA to enrich for sequences that resemble a given target such that upon sequencing, fewer sequencing reads are required to fully enumerate all variants in the complex mixture with high coverage, which would otherwise be most costly and time-consuming for a non-enriched sample.

To “mine” physical DNA samples for nucleic acid sequences that code for proteins homologous to a target of interest, one can perform steps listed. The methods provided herein use target enrichment for the purposes of assembling a superior MSA for protein structure prediction. No protein structure prediction software uses physical, experimental methodology for constructing an MSA. The bigger and more diverse an MSA is, the higher quality DCA that can be performed, the more precise the generated contact map estimation, and the more accurate the 3D structure prediction.

There are multiple target enrichment strategies, but one in particular, called Scodaphoresis, is particularly attractive for mining homologs from physical samples. Provided herein is modified scodaphoresis for target enrichment of divergent homologs, where the design of probe sequences and target enrichment conditions is intentionally manipulated to enrich as many sequence variants as possible with relaxed stringency.

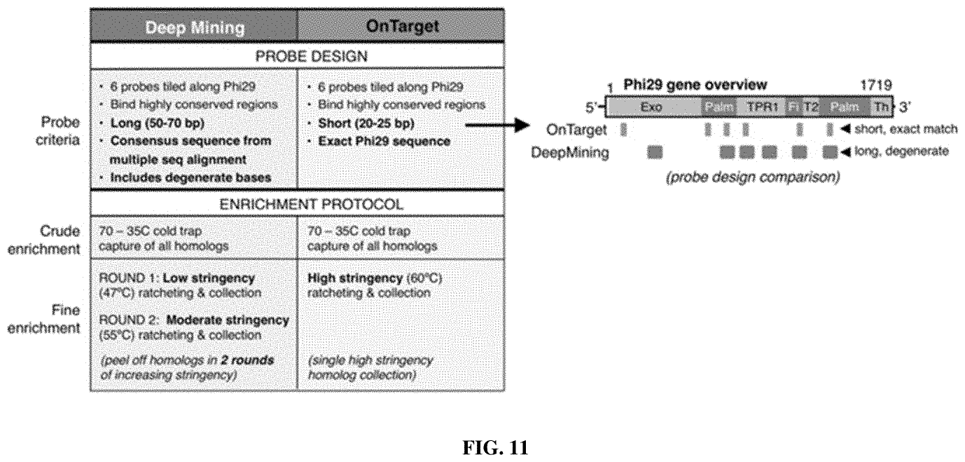

Below is a description of the methods used to enrich Phi29-like genes from a soil sample by scodaphoresis, as well as figures describing the data and analyzed results.
1. Environmental DNA was extracted from wet soil at 351A New Whitfield St, Guilford, Conn. 06437 using the PowerSoil DNeasy Pro kit. The manufacturer's instructions were followed.
2. Soil DNA was simultaneously fragmented down to 1-3 kb and appended with adapters using the tagmentation method.
3. 8 known Phi29 homologs (2 kb in length) that range in Phi29 homology from 40-100% were spiked into the tagmented soil DNA sample at low abundance (1:1000 mass ratio) >these serve as positive controls for enrichment and enable quantification of enrichment as a function of % homology.
4. Spiked soil sample was enriched for Phi29 using two different scodaphoresis methodologies (see FIG. 11), while a control sample was not enriched.
5. Scodaphoresis consisted of the following general steps:
   a. Capture tagmented, spiked soil sample in separation medium containing immobilized Phi29 probe set. “Off target” (highly mobile) sequences will flow through the separation medium and be removed at this stage.
   b. Release previously low mobility, gel-immobilized, enriched sequences by a step change elevation in the temperature.
      i. Recovery of enriched sequences that are highly mobile is possible at elevated temperature by their electrophoresis out of the gel-like matrix.
      ii. Enriched sequences can be recovered from an extraction port.
      iii. Program a series of gradual step changes in temperature to selectively release one or more enriched nucleic acid sequences according to their hybridization binding energy to the immobilized phase.
      iv. With perpendicular electric fields, switch directions of the electrophoresis driving force to run enrichment in series where the low-mobility material that remains in the gel after one round of enrichment is the starting material for a subsequent round.
      v. Use of dynamic, rotating electric fields to drive synchronous coefficient of drag alteration (SCODA) electrophoresis to finely differentiate nucleic acid variants according to slight differences in their mobilization at different temperatures.
6. Library prep (SMRTBell Template Prep kit 1.0) and long-read, circular consensus PacBio sequencing.
7. Long read, circular consensus sequencing and analysis on enriched v. unenriched samples.

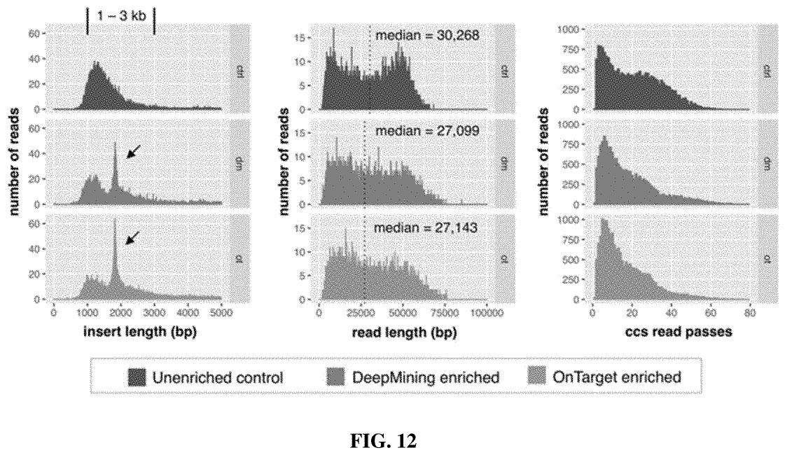

Across all samples, insert sizes were 1-3 kb (as expected from tagmentation results) and median read lengths approached 30 kb. That means that circular consensus was performed on 10-20 passes for very high accuracy reads (FIG. 12)

Interestingly, the insert size distribution changed after enrichment such that a strong peak at 2 kb emerged, as marked by arrows in FIG. 12. This reflects that the 2 kb positive control homologs that were spiked into the soil sample were so strongly enriched that they represent a large fraction of the inserts and show up prominently at a single length in the insert length distribution.

Next, it was determined what kinds of protein-coding sequences were in the unenriched soil DNA sample and how the distribution of those proteins changed after enrichment. For each 1-3 kb circular consensus sequence, all 6 frames were translated and identified the presence of conserved protein domains in the resulting open reading frames. Prior to enrichment, the most abundant protein domains are related to signaling and transport across the membrane among other putative functions. DNA polymerases of the family B type represented just 0.03% of the protein domains in the unenriched sample and were only present in the unenriched due to positive control Phi29 homologs spike-in—no Phi29 homologs outside of spiked-in controls were identified in the unenriched sample.

After enrichment, family B DNA polymerases represent 44% of the protein domains identified among the OnTarget and DeepMining enriched samples, reflecting a strong level of enrichment at the protein domain level (˜1000×).

By spiking in 8 different known Phi29 homologs of varying % homology to Phi29 at low abundance in the unenriched sample, fold changes for individual homologs were quantified and functional differences between the OnTarget and DeepMining strategies were determined.

Importantly, all 8 homologs were detected in both enrichment samples. It was found that enrichment of the homologs varied—from as low as—fold enrichment of AP50 (42% homology to Phi29) by DeepMining to >1400 fold enrichment of B103 (75% homology to Phi29) by OnTarget enrichment.

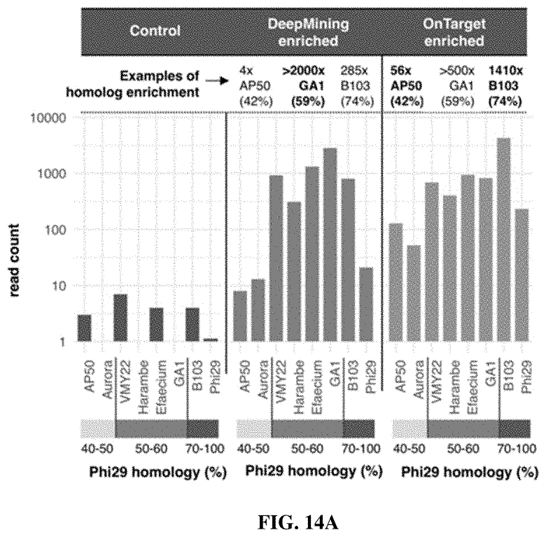

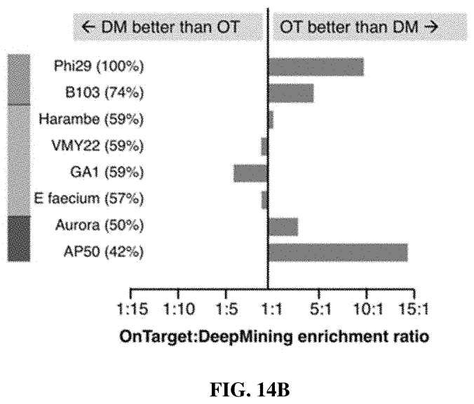

When the enrichment performance of OnTarget and DeepMining were compared head-to-head, an interesting trend was observed (FIG. 14). OnTarget excelled at enriching sequences with high (75-100%) homology to Phi29 (5-10-fold better than DeepMining), and it also, surprisingly outperformed DeepMining for the lowest homology sequences. DeepMining was slightly superior to OnTarget (1.5-5-fold better) at enriching 3 of the 4 medium homology sequences.

Because the intention of enrichment is for new homolog discovery, it was desirable to look for the presence of Phi29 homologs beyond those that were intentionally added as spike-in controls.

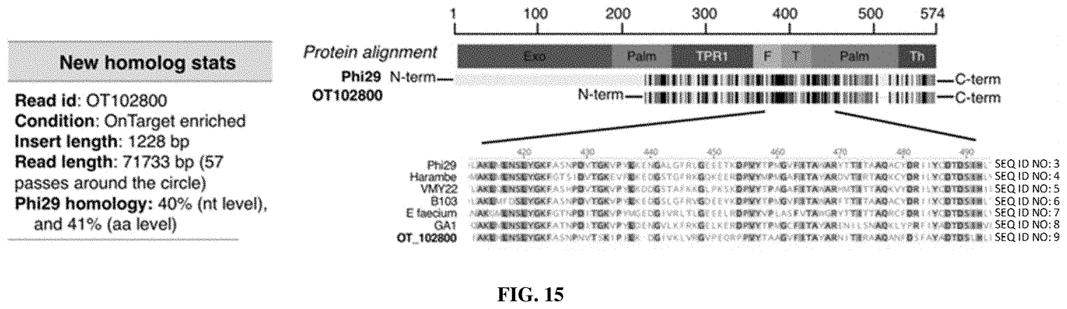

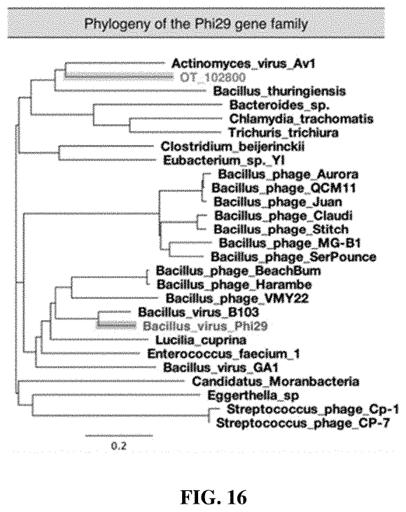

One new Phi29 homolog—OT102800 (FIG. 15)—was identified among the OnTarget enriched sequences and added to the Phi29 gene family phylogenetic tree (FIG. 16). Finding one new homolog from 1 μg of starting soil DNA validated this approach.

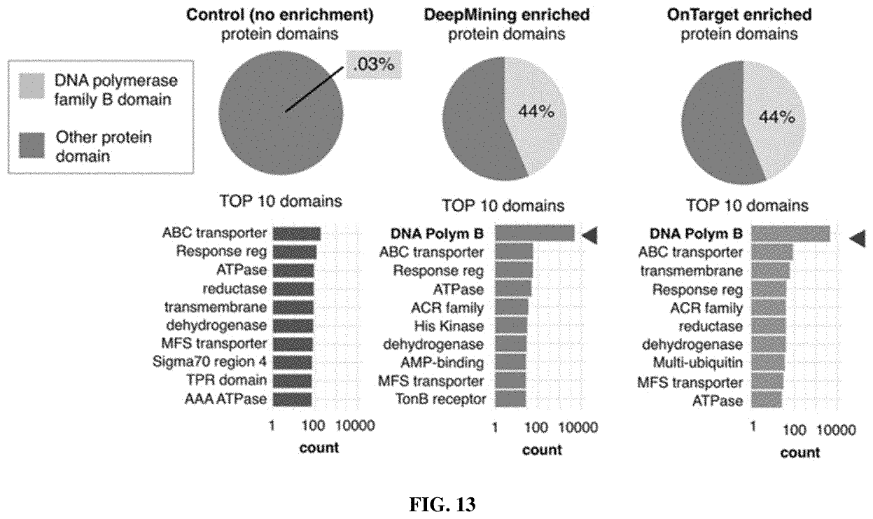

As described by FIGS. 12 and 13, the new homolog is 40% homologous to Phi29 at the nucleotide level and once translated, the environmental fragment aligns to Phi29 from the Palm region through the end of the polymerase. Although the homolog was identified from a single sequencing read, accuracy for the molecule was high (57 ccs passes).

Primers may be designed to amplify OT102800 directly from the original soil sample by PCR to confirm its presence and determine the full-length sequence.

What is claimed is:

1. A method of in silico mining for new homologs of a protein of interest, the method comprising:
producing an initial protein homolog sequence database (DBinit) for the protein of interest;
generating a representative reference database (DBrep) of putative protein homolog sequences by eliminating multiple sequences in the DBinit that share at least 75% identity;
screening a metagenomic sequencing read archive, optionally a sequencing read archive, using the DBrep as a query to identify datasets of sequencing reads, and optionally ranking the datasets to determine which are most likely to contain the highest number of true homologs;
aligning the DBrep to the sequencing reads, optionally all sequencing reads, from a given metagenomic dataset;
assembling the aligned sequencing reads into contigs;
translating open reading frames (ORFs) of the contigs into protein sequences having greater than a cutoff fraction of the length of the average DBrep protein sequence;
aligning the translated protein sequences with the DBrep protein sequences and identifying new putative protein homolog sequences, and optionally adding the new putative protein homolog sequences to the DBinit to produce an enhanced protein homolog sequence database (DBenhanced).

2. The method of claim 1, wherein the producing a protein homolog sequence database includes searching protein family databases for proteins containing a conserved protein domain.

3. The method of claim 1, wherein the producing a protein homolog sequence database includes searching protein sequence databases using pairwise or hidden Markov model (HMM)-based alignment.

4. The method of claim 1, further comprising assessing completeness of the DBinit by aligning a known non-redundant protein reference database and the DBinit, optionally using a protein alignment tool adapted for large query sets, and searching for additional homologs of the protein of interest.

5. The method of claim 1, wherein the DBprep is generated by clustering the DBinit at 90% using a clustering algorithm.

6. The method of claim 1, wherein the aligning the DBrep to sequencing reads of each of the SRA datasets comprises aligning the DBrep to a sampling of reads/read-pairs from every whole-genome metagenomic run in the SRA, optionally wherein the sampling size is about 100,000 reads.

7. The method of claim 1, further comprising quality control steps to remove unassembled reads from the metagenomic datasets.

8. The method of claim 1, wherein the translating comprises translating six ORFs of the contigs.

9. The method of claim 1, further comprising quality control steps to validate the putative protein homolog sequences as true protein homolog sequences, which are then optionally added to the DBenhanced.

10. The method of claim 1, further comprising target protein enrichment.

11. The method of claim 1, further comprising generating a representative multiple sequence alignment (MSA) based on the DBenhanced.

12. A target enrichment method comprising:
providing a list of putative protein homolog sequences of a protein of interest from a multiple sequence alignment (MSA) of sequences homologous to the protein of interest;
contacting a sample comprising DNA with probes to produce probes bound to DNA, wherein the probes are designed to hybridize, optionally with low stringency, to the nucleotide sequences of the putative protein homolog sequences, and wherein the probes are immobilized on a substrate that optionally includes a separation medium;
optionally selectively removing from the substrate probes that are not bound to DNA;
sequencing the DNA bound to the probes to produce sequencing reads;
aligning the sequencing reads to the MSA and assembling contigs from any sequencing reads that are shorter than the full-length sequence of the protein;
translating open reading frames (ORFs) from the contigs to generate new putative protein homolog sequences, and optionally validating the new putative protein homolog sequences as true protein homolog sequences; and
optionally adding the new putative protein homolog sequences to the MSA to produce an enriched MSA.

13. The method of claim 12, further comprising executing on the MSA an algorithm for deducing direct correlation, optionally wherein the algorithm is a Direct Coupling Analysis (DCA) algorithm.

14. The method of claim 12, further comprising performing feature extraction using the enriched MSA for a co-evolution-based protein structure prediction model.

15. A computer readable medium on which is stored a computer program which, when implemented by a computer processor, causes the processor to:
produce an initial protein homolog sequence database (DBinit) for the protein of interest;
generate a representative reference database (DBrep) of putative protein homolog sequences by eliminating multiple sequences in the DBinit that share at least 75% identity;
screen the sequencing read archive (SRA) using the DBrep as a query to identity datasets of sequencing reads, and optionally rank the datasets to determine which are most likely to contain the highest number of true homologs.

16. The computer readable medium of claim 15, wherein the computer program further causes the processor to:
align the DBrep to sequencing reads of the SRA datasets to identify hit reads;
assemble hit reads into contigs;
translate open reading frames (ORFs) of the contigs into protein sequences having greater than a cutoff fraction of the length of the average DBrep protein sequence;
align the translated protein sequences with the DBrep protein sequences and identifying new putative protein homolog sequences, and optionally add the new putative protein homolog sequences to the DBinit to produce an enhanced protein homolog sequence database (DBenhanced).

17. A computer readable medium on which is stored a computer program which, when implemented by a computer processor, causes the processor to:
align sequencing reads to a multiple sequence alignment (MSA) and assembling contigs from any sequencing reads that are shorter than a full-length sequence of the protein;
translating open reading frames (ORFs) from the contigs to generate new putative protein homolog sequences; and
add the new putative protein homolog sequences to the MSA to produce an enriched MSA.

18. A computer implemented method of mining for new homologs of a protein of interest, the method comprising:
producing an initial protein homolog sequence database (DBinit) for the protein of interest;
generating a representative reference database (DBrep) of putative protein homolog sequences by eliminating multiple sequences in the DBinit that share at least 75% identity;
screening a metagenomic sequencing read archive using the DBrep as a query to identity datasets of sequencing reads, and optionally ranking the datasets to determine which are most likely to contain the highest number of true homologs;
aligning the DBrep to sequencing reads of the metagenomic datasets;
assembling the aligned sequencing reads into contigs;
translating open reading frames (ORFs) of the contigs into protein sequences having greater than a cutoff fraction of the length of the average DBrep protein sequence;
aligning the translated protein sequences with the DBrep protein sequences and identifying new putative protein homolog sequences, and optionally adding the new putative protein homolog sequences to the DBinit to produce an enhanced protein homolog sequence database (DBenhanced).

19. The computer implemented method of claim 15, further comprising assessing completeness of the DBinit by aligning a known non-redundant protein reference database and the DBinit, optionally using a protein alignment tool adapted for large query sets, and searching for additional homologs of the protein of interest.

20. A computer implemented iterative homolog discovery method comprising:
(a) performing the method of claim 11 to produce an enhanced multiple sequence alignment (MSA);
(b) inputting results new putative protein homolog sequences obtained from a target enrichment method, wherein the DNA sample has been identified using metadata for metagenomic SRA samples with positive homolog identification;
(c) adding the new putative protein homolog sequences to the enhanced MSA; and
(d) optionally repeating the steps (a)-(c) iteratively.
